(19) United States
(12) Patent Application Publication
(10) Pub. No.: US 2009/0000970 A1
(43) Pub. Date: Jan. 1, 2009

(54) Prepackaged Intraluminal Medical Device

(75) Inventors: Jerome Bordeau, Condeau (FR); Remi Collin, Saint Hilaire sur Erre (FR)

Correspondence Address: YOUNG & THOMPSON, 209 Madison Street, Suite 500, ALEXANDRIA, VA, 22314, US

(73) Assignee: B. Braun Medical SAS, BOULOGNE-BILLANCOURT (FR)

(21) Appl. No.: 12/161,589

(22) PCT Filed: Jan. 19, 2007
(86) PCT No.: PCT/FR07/00106

§ 371 Date: Jul. 21, 2008

(30) Foreign Application Priority Data
Jan. 20, 2006 (FR) 06/00544

(51) Int. Cl. A61B 19/02; A61M 25/00

(52) U.S. Cl. 206/364; 604/523

(57) ABSTRACT

An intraluminal device intended to be introduced into a passage within the body, includes a tube (10) having one end (12) intended to be introduced into the bodily passage, a second end (14) connected to a receiving member (16) and a part for introduction into the bodily passage, and a wrapper (20) formed of two sheets of plastic which is intended to form an opening for the passage of the first end (12) of the tube and has two parts for preferential parting (22, 24) for forming an opening, the superposed sheets of the wrapper (20) forming three parts for grasping (26, 28, 30) positioned on each side of the parts for preferential parting (22, 24).

This invention relates to an intraluminal medical device that is designed to be inserted into a bodily duct, and, more specifically, such a device that is placed in a package that has the shape of a specifically adapted envelope.

Intraluminal medical devices are now used that comprise a thin tube that is rigid enough not to fold on itself and flexible enough to follow the curves of a bodily duct, for example the urethra of a patient, whereby the tube has a first end, designed to be inserted into the bodily duct, a second end connected to a receiving element, and an insertion part between the first end and a point placed between the two ends, whereby the insertion part is designed to be inserted at least partially into the bodily duct. These devices are each arranged in an envelope that is formed by two plastic sheets that are arranged on top of one another, made integral with one another, at least on sides that are essentially parallel to the thin tube, by at least one connecting strip with a width that is at least equal to a minimum value and designed to enclose in a sealed manner, before use, the insertion part of the thin tube, whereby the envelope is designed to form an opening for the passage of the first end of the tube outside of the envelope so that it is inserted into the bodily duct and that has for this purpose a preferred separation section for the formation of this opening.

The thin tube part that has to be inserted into the bodily duct varies more or less from one patient to the next. In addition, since the tube is in general equipped with a lubricant and so that, after opening the envelope, the state of sterility of the tube part to be inserted is preserved as much as possible, it is desirable that only this part be detached from the envelope. In addition, it is desirable that the envelope to be tearable at different points along its length to detach the end to be connected, to detach the insertion end and/or to separate the envelope at an intermediate point of its length when the user so desires.

The formation on the envelope of preferred separation sections, formed by, for example, thinning the material of the envelope and indicated by a transverse line, is therefore already known. The user grasps the envelope on both sides of the line and separates his hands to tear it. This method exhibits drawbacks because it most often makes it necessary to squeeze the tube; this gives rise to producing a local void in its lubricant, and handling is difficult—in particular for individuals with reduced dexterity—and also creates the risk that the lubricant may shift.

According to the document EP-372 721, a packaging of a catheter that is equipped with an optical fiber is also known. This packaging delimits two compartments, one of which is to remain sterile while the other is open. The packaging comprises a base that delimits two compartments that are connected by a narrow part and an oriented-polymer envelope sheet that covers the base of the device. A portion of the sheet can be removed from a compartment, and, for this purpose, it can be separated preferably along a line that is arranged between the two compartments. The opening is made by detaching the sheet that is attached to the base of the compartment and not by tearing the sheet and the base.

The object of the invention is the production of an intraluminal medical device, equipped with a packaging envelope, formed by two sheets that are arranged on top of one another, in which the preferred separation of the two sheets at one or more selected points of the envelope can be carried out in a very convenient manner.

More specifically, the invention uses several means that, separately or combined, facilitate the use of such intraluminal medical devices. These means are, on the one hand, the use of a particular material, composed of an oriented polymer, for the formation of two sheets of the envelope, and, on the other hand, the formation of specific gripping parts, in particular the specific arrangement of gripping parts and preferred separation sections.

Thus, according to the invention, the application of a force between a grasping zone and a gripping part causes separation to take place at a determined point. Obtaining a separation at another point requires the grasping of a grasping zone and another gripping part.

More specifically, the invention relates to an intraluminal medical device that is designed to be inserted into a bodily duct, comprising a thin tube that is rigid enough not to fold on itself and flexible enough to follow the curves of a bodily duct, whereby the tube has a first end, designed to be inserted into the bodily duct, a second end connected to a receiving element, and an insertion part between the first end and a point arranged between the two ends, whereby the insertion part is designed to be inserted at least partially into the bodily duct, and an envelope that is formed by two plastic sheets that are arranged on top of one another, made integral with one another, at least on the sides that are essentially parallel to the thin tube, by at least one connecting strip with a width that is at least equal to a minimum value, and designed to enclose in a sealed manner, before use, the insertion part of the thin tube, whereby the envelope is designed to form an opening for the passage of the first end of the tube outside of the envelope for its insertion into the bodily duct and that has for this purpose at least one preferred separation section for the formation of an opening; according to the invention, the plastic material of the sheets of the envelope is an oriented polymer, and the direction of orientation of the oriented polymer is essentially transverse to the elongation direction of the tube of the intraluminal device.

Preferably, the preferred separation section comprises, in at least one connecting strip, a slot that reduces the width of the connecting strip and forms an initiator for tearing the two plastic sheets.

In an advantageous embodiment, the sheets of the envelope that are arranged on top of one another form at least one gripping part and a grasping zone, whereby the gripping part and the grasping zone are arranged on both sides of the preferred separation section.

In a variant, the envelope comprises at least two preferred separation sections and at least two gripping parts, of which one is arranged between two preferred separation sections, and the other outside of the envelope segment between the two preferred separation sections.

It is advantageous for a gripping part at least to be offset laterally toward the outside along the envelope. In one example, the envelope comprises two end gripping parts and one intermediate gripping part offset laterally toward the outside along the envelope. It is then advantageous that an initiator for tearing the two plastic sheets be formed close to the intermediate gripping part that is offset laterally.

Preferably, the plastic material of the sheets that are arranged on top of one another is selected from among the oriented polyethylene and the bi-oriented polyethylene.

In one embodiment, the receiving element is a pocket attachment piece.

In another embodiment, the receiving element is a pocket.

Preferably, the thin tube carries a lubricant material at least on the surface of the insertion part.

The invention also relates to the application of an intraluminal device as defined in the urinary catheterization of a human body.

The document EP-372 721 neither describes nor suggests the fact that the device part (catheter) that is removed is that which is to be inserted into the bodily duct, nor the fact that opening by tearing oriented-polymer sheets is carried out by opening the cavity that is delimited between the two sheets, since it defines only the separation of a sheet bonded to a base.

In this report, “grasping zone” is the term used to refer to an extended part of the device that can be held clamped between two fingers at a point on the envelope that is normally inside the connecting strip. “Gripping part” is the term used to refer to an extended part of the envelope that can be held clamped between two fingers at a point on the envelope that is normally outside the connecting strip and that is formed by an extension of the sheets of the envelope.

In this report, the term “sealed” used in reference to the plastic sheets indicates that the latter do not allow liquids to pass, even though they may have a certain gas permeability, in particular for allowing sterilization.

Other characteristics and advantages of the invention will be better understood from reading the following description of embodiments, given with reference to the accompanying drawings, in which:

FIG. 1 is a diagrammatic view of an intraluminal medical device according to the invention, allowing the description of characteristics of the invention;

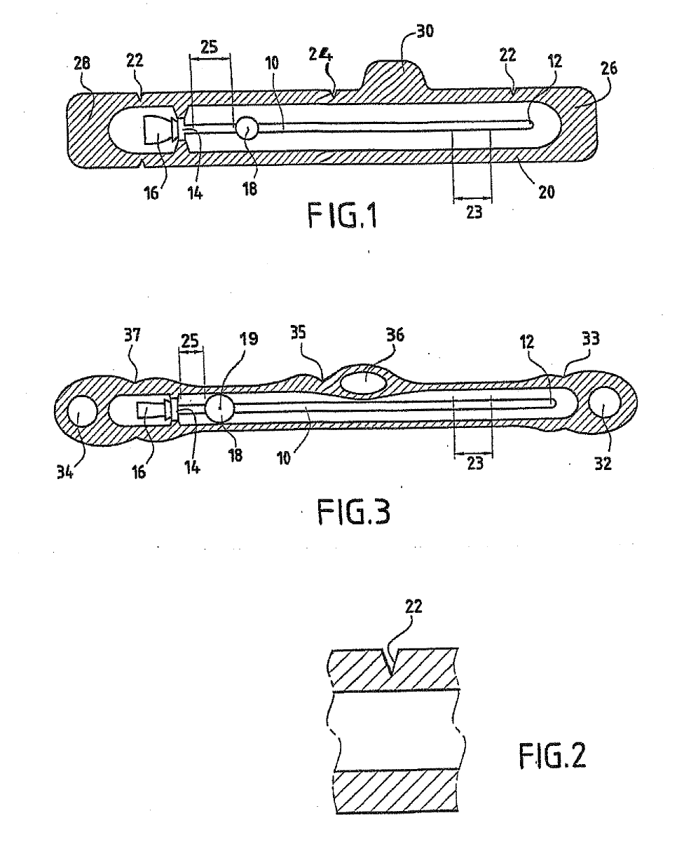

FIG. 2 shows a portion of FIG. 1 in enlarged form;

FIG. 3 illustrates a useful variant for the production of devices according to the invention; and

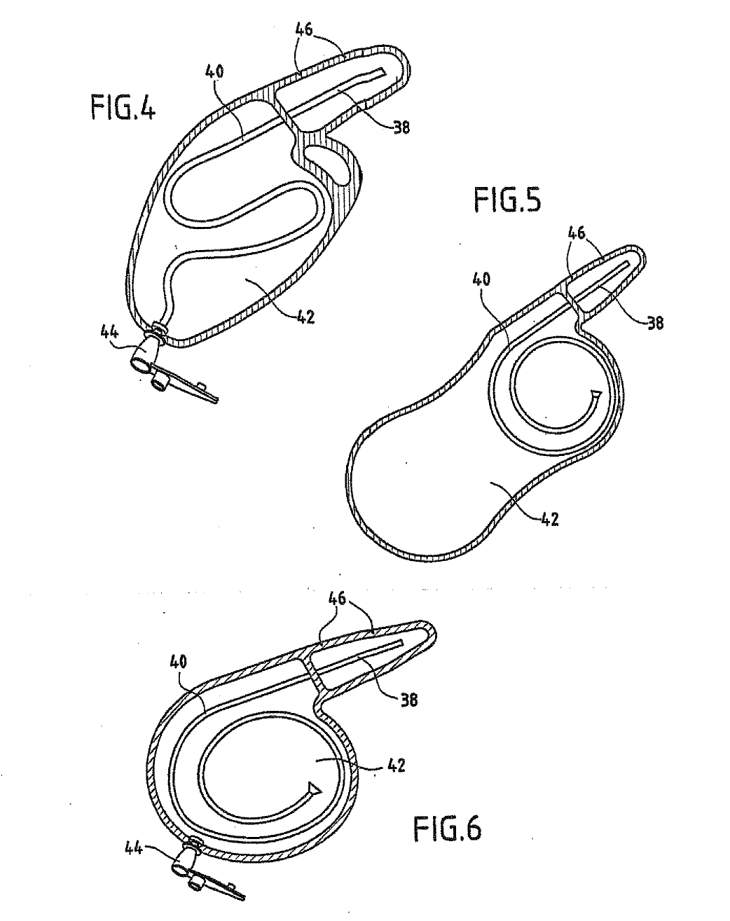

FIGS. 4, 5 and 6 show three variant embodiments of pocket devices that have only one of the improvements according to the invention.

FIG. 1 shows an intraluminal medical device according to an embodiment of the invention that combines the two aspects of the invention. More specifically, it comprises a tube 10 that is designed to be inserted into a bodily duct, such as the urethra, and carries a lubricant to the outside. The first end 12 of the tube is designed to be inserted into the bodily duct, and the other end 14 is connected to a receiving element 16, for example, a pocket attachment piece. The tube preferably bears an air-hole system 18 that is designed to allow the ventilation of the envelope. This air-hole, for example, comprises a perforation 19 that is adjacent to a gas-permeable element but not to a liquid-permeable element, for example a pellet made of foam or a gas-permeable film or a non-woven material. This element can be bonded or welded to a sheet. Although this air-hole system has been shown on a part that is detached from the sheet, it may be located at the connection of the piece 16 and the tube 10, close to the end 14.

The element 16 is only one example, however, because the tube 10 can also be directly connected to a receiving pocket, as suggested by FIGS. 5 and 6.

In the embodiment of FIG. 1, an envelope consists of two plastic sheets that are welded at the periphery of the envelope so that the tube 10 is enclosed in a cavity.

Of course, according to current techniques, the plastic sheets that are used may have a certain permeability so that they allow the sterilization of the tube, according to conventional techniques, for example by use of a vacuum and ethylene oxide.

The lateral connecting strips of the envelope have slots such as 22, 24 to initiate tearing, having, for example the shape of a V or a simple incision. According to the known technique, at the desired preferred separation, the plastic sheets can have a reduced thickness that thus facilitates tearing.

A first advantageous characteristic of the invention is the formation of plastic sheets into an oriented polymer whose direction of orientation is transverse to the length of the tube 10 and of the envelope shown in FIG. 1, so that the envelope is easily cut in a transverse direction. For this purpose, the oriented polymer is preferably a polyolefin, advantageously oriented polyethylene or bi-oriented polyethylene.

A second characteristic of the invention is the use of gripping parts such as 26, 28, and 30. These parts are formed by extended parts of the plastic sheets, allowing a firm grasp by clamping between two fingers.

In the embodiment of FIG. 1, when the device is grasped between two fingers of one hand by the gripping part 26 and between two fingers of the other hand in the grasping zone 23 and when a separation force is applied, the envelope is torn in a transverse direction from the slot 22. When the device is grasped between two fingers of one hand by the gripping part 28 and between two fingers of the other hand in the grasping zone 25 and when a separation force is applied, the envelope is torn in a transverse direction from the other slot 22. When the device is grasped between two fingers of one hand by the gripping part 30 and between two fingers of the other hand in the grasping zone 25, and when a separation force is applied, the envelope is torn in a transverse direction from the slot 24 all the more easily since the axis of application of the force is not combined with the longitudinal axis and the force is concentrated on the slot 24.

This selection of the preferred separation section by simple selection of the grasping zones and gripping parts is a first advantageous characteristic of the invention.

FIG. 3 shows another embodiment in which the gripping parts comprise orifices such as 32, 34, and 36 that allow the passage of a finger. In this way, the applied force can be very high, and the handling is very simple. Following the orifice into which pass a finger of one hand and the zone for gripping by two fingers of the other hand, the envelope separates at different points, indicated by the slots 33, 35, and 37.

Of course, the number of gripping parts, comprising flaps such as 30 or orifices such as 32, 36, is not limited. However, a larger number runs the risk of appearing complicated to the user, who, in contrast has very little problem understanding the use of three gripping parts distributed along the envelope.

FIGS. 4 to 6 show three embodiments in which the tube or catheter 38 is connected to a flexible tube 40 that is arranged in a pocket 42. The pocket can comprise an exit plug 44 and the tube 40 can be connected to the latter, as indicated in FIG. 4. The tube can also be simply wound on itself as indicated in FIGS. 5 and 6.

According to the invention, the sheets of these pockets are formed by oriented polymer such that a tear that is initiated in a slot 46 passes through the front part of the device that contains the catheter 38 and allows access to the latter.

The plastic material of the sheets that are arranged on top of one another is advantageously selected from among the polyolefins, such as the polyethylene that is oriented in a single direction or in multiple directions; when it is oriented in multiple directions, the elongation coefficients and therefore the orientation coefficients are not the same in the different directions and there is an overall effect resulting from the preferred orientation, preferably aligned according to the width of the envelope.

1. Intraluminal medical device designed to be inserted into a bodily duct, comprising a thin tube (10) that is rigid enough not to fold on itself and flexible enough to follow the curves of a bodily duct,
whereby the tube (10) has a first end (12), designed to be inserted into the bodily duct, a second end (14) that is connected to a receiving element (16), and an insertion part between the first end and a point placed between the two ends, whereby the insertion part is designed to be inserted at least partially into the bodily duct, and
an envelope (20) that is formed by two plastic sheets that are arranged on top of one another, made integral with one another, at least on the sides that are essentially parallel to the thin tube (10), by at least one connecting strip with a width that is at least equal to a minimum value and designed to enclose in a sealed manner, before use, the insertion part of the thin tube (10),
whereby the envelope (20) is designed to form an opening for the passage of the first end (12) of the tube outside of the envelope (20) so that it is inserted into the bodily duct and that has for this purpose at least one preferred separation section for the formation of an opening,
characterized in that the plastic material of the sheets of the envelope (10) is an oriented polymer, and
the direction of orientation of the oriented polymer is essentially transverse to the elongation direction of the tube of the intraluminal device.

2. Intraluminal device according to claim 1, wherein the preferred separation section (22, 24; 33, 35, 37, 46) comprises, in at least one connecting strip, a slot that reduces the width of the connecting strip and forms an initiator for tearing the two plastic sheets.

3. Intraluminal device according to claim 1, wherein the sheets of the envelope (20) that are arranged on top of one another form at least one gripping part (26, 28, 30; 32, 34, 36) and a grasping zone (23, 25), whereby the gripping part and the grasping zone are arranged on both sides of the preferred separation section.

4. Intraluminal device according to claim 3, wherein the envelope (20) comprises at least two preferred separation sections (22, 24; 33, 35, 37, 39) and at least two gripping parts (26, 28, 30; 32, 34, 36), one of which (30; 36) is arranged between the two preferred separation sections and the other outside of the envelope segment between the two preferred separation sections.

5. Intraluminal device according to claim 3, wherein at least one gripping part (30; 36) is offset laterally toward the outside along the envelope (20).

6. Intraluminal device according to claim 3, wherein the envelope (20) comprises two end gripping parts (32, 34) and one intermediate gripping part (30, 36) that is offset laterally toward the outside along the envelope (10).

7. Intraluminal device according to claim 6, wherein an initiator for tearing two plastic sheets is formed close to the intermediate gripping part (36) that is offset laterally.

8. Intraluminal device according to claim 1, wherein the plastic material of the sheets that are arranged on top of one another is selected from among the oriented polyethylene and the bi-oriented polyethylene.

9. Intraluminal device according to claim 1, wherein the receiving element is a pocket attachment piece (16).

10. Intraluminal device according to claim 1, wherein the receiving element is a pocket.

11. Intraluminal device according to claim 2, wherein the sheets of the envelope (20) that are arranged on top of one another form at least one gripping part (26, 28, 30; 32, 34, 36) and a grasping zone (23, 25), whereby the gripping part and the grasping zone are arranged on both sides of the preferred separation section.

12. Intraluminal device according to claim 4, wherein at least one gripping part (30; 36) is offset laterally toward the outside along the envelope (20).

13. Intraluminal device according to claim 4, wherein the envelope (20) comprises two end gripping parts (32, 34) and one intermediate gripping part (30, 36) that is offset laterally toward the outside along the envelope (10).

14. Intraluminal device according to claim 5, wherein the envelope (20) comprises two end gripping parts (32, 34) and one intermediate gripping part (30, 36) that is offset laterally toward the outside along the envelope (10).
